(19) United States
(12) Patent Application Publication
(10) Pub. No.: US 2022/0293245 A1
(43) Pub. Date: Sep. 15, 2022

(54) VOICE CONTROLLED MEDICAL DIAGNOSIS SYSTEM

(71) Applicant: GE Precision Healthcare LLC, Wauwatosa, WI (US)

(72) Inventor: Jerome Knoplioch, Neuilly Sur Seine (FR)

(21) Appl. No.: 17/751,012

(22) Filed: May 23, 2022

Related U.S. Application Data
(63) Continuation of application No. 16/701,950, filed on Dec. 3, 2019.

(51) Int. Cl. G16H 30/20; G16H 15/00; G06T 7/00

(52) U.S. Cl. CPC G16H 30/20; G16H 15/00; G06T 7/0012; G06T 2207/20081

(57) ABSTRACT

Systems and methods for synchronizing medical image analysis and reporting are provided, included in a voice controlled medical diagnosis system. The method includes receiving an examination type selection. The method includes selecting a viewing context (e.g., hanging protocol and/or image analysis tools) applied by an image viewer to medical images based on the examination type selection. The method may include selecting a reporting template based on the examination type selection. The method includes receiving a user analysis input (e.g., measurement and/or annotation) with reference to one of the medical images presented via the image viewer at a display system according to the viewing context. The method includes generating and presenting an image object having the user analysis input in the medical image via the image viewer at the display system. The method includes generating a report object corresponding to the image object.

### CROSS REFERENCE TO RELATED APPLICATIONS

This application is a continuation of patent application Ser. No. 16/701,950, entitled “Method and System for Synchronizing Medical Image Analysis and Reporting,” filed Dec. 3, 2019, which is herein incorporated by reference in its entirety for all purposes.

### FIELD

Certain embodiments relate to medical imaging analysis and reporting. More specifically, certain embodiments relate to a method and system for synchronizing medical image analysis and reporting and voice controlled medical diagnosis system.

### BACKGROUND

Medical imaging examinations typically involve the acquisition of medical image data, analysis of the acquired medical image data, and the generation of a report documenting the findings of the medical imaging examination. The medical professional performing the examination typically analyzes the acquired medical image data to perform measurements and add annotations. The medical professional may subsequently prepare the report by reviewing the measurement and annotation information from the analyzed images and adding the information to the report. However, the separate workflows for analyzing the medical images and preparing the report may be inefficient and involve redundant steps or data entries. Moreover, information created during the image analysis phase may be inadvertently missed and not provided in the report.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for synchronizing medical image analysis and reporting, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary medical workstation that is operable to provide synchronized medical image analysis and reporting, in accordance with various embodiments.

FIG. 2 is a flow chart illustrating exemplary steps that may be utilized for synchronizing medical image analysis and reporting, in accordance with various embodiments.

### DETAILED DESCRIPTION

Certain embodiments may be found in a method and system for synchronizing medical image analysis and reporting. Various embodiments have the technical effect of creating an image object in medical image data and a corresponding report object in a report template in response to a user analysis input, such as a measurement instruction or annotation. Aspects of the present disclosure have the technical effect of facilitating dictation of measurements and/or annotations simultaneously in both an image analysis application and a reporting application template. Certain embodiments have the technical effect of selecting one or both of a viewing context (e.g., hanging protocol, analysis tools, etc.) in an image viewer and a reporting template in a reporting application based on a selected examination type. Various embodiments have the technical effect of associating image objects in medical image data with report objects in a report template such that updates to either of the objects updates both of the objects.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. For example, as used herein the term “image” is used to refer to ultrasound images, magnetic resonance imaging (MRI) images, computed tomography (CT) images, and/or any suitable medical image. Further, with respect to ultrasound imaging, for example, the term “image” may refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MA/I, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

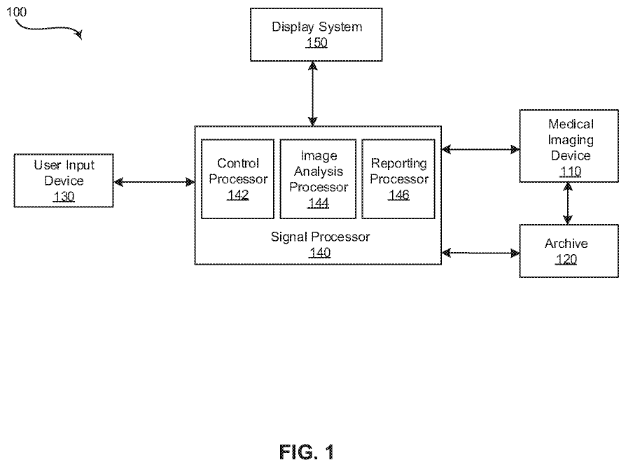

FIG. 1 is a block diagram of an exemplary medical workstation 100 that is operable to provide synchronized medical image analysis and reporting, in accordance with various embodiments. Referring to FIG. 1, the medical workstation 100 comprises a display system 150, a signal processor 140, and a user input device 130. The medical workstation 100 may include and/or be communicatively coupled to an archive 120 and a medical imaging device 110. Components of the medical workstation 100 may be implemented in software, hardware, firmware, and/or the like. The various components of the medical workstation 100 may be communicatively linked. Components of the medical workstation 100 may be implemented separately and/or integrated in various forms. For example, the display system 150 and the user input device 130 may be integrated as a touchscreen display. As another example, the workstation 100 may be communicatively coupled to one or more servers operable to perform at least some of the processing of the signal processor 140 as described below.

The display system 150 may be any device capable of communicating visual information to a user. For example, a display system 150 may include a liquid crystal display, a light emitting diode display, and/or any suitable display or displays. The display system 150 can be operable to display information from the signal processor 140 and/or archive 120, such as medical images, labeling tools, automated analysis tools, annotations, measurements, reports, or any suitable information.

The user input device 130 may include any device(s) capable of communicating information from a user and/or at the direction of the user to the signal processor 140 of the medical workstation 100, for example. For example, the user input device 130 may include a touch panel, dictation device with voice recognition, button(s), a mousing device, keyboard, rotary encoder, trackball, camera, and/or any other device capable of receiving a user directive.

The archive 120 may be one or more computer-readable memories integrated with the medical workstation 100 and/or communicatively coupled (e.g., over a network) to the medical workstation 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 120 may include databases, libraries, sets of information, or other storage accessed by and/or incorporated with the signal processor 140, for example. The archive 120 may be able to store data temporarily or permanently, for example. The archive 120 may be capable of storing medical image data, reports, data generated by the signal processor 140, and/or instructions readable by the signal processor 140, among other things. In various embodiments, the archive 120 stores medical images, medical image measurements, medical image annotations, report templates, reports, and/or instructions for performing measurements, automated analysis, report template selection, and/or report generation, among other things.

The signal processor 140 may be one or more central processing units, microprocessors, microcontrollers, and/or the like. The signal processor 140 may be an integrated component, or may be distributed across various locations, for example. The signal processor 140 comprises a control processor 142, an image analysis processor 144, and a reporting processor 146, and may be capable of receiving input information from a user input device 130 and/or archive 120, generating an output displayable by a display system 150, and manipulating the output in response to input information from a user input device 130, among other things. The signal processor 140, control processor 142, image analysis processor 144, and/or reporting processor 146 may be capable of executing any of the method(s) and/or set(s) of instructions discussed herein in accordance with the various embodiments, for example.

The signal processor 140 may include an image analysis processor 144 that comprises suitable logic, circuitry, interfaces and/or code that may be operable to display medical images (e.g., image viewer) and analysis tools for analyzing the medical images. For example, the image analysis processor 144 may retrieve a hanging protocol and image analysis tools associated with a particular viewing context in response to an examination selection provided by a user via the user input device 130 and/or control processor 142. The hanging protocol may include display format and viewing instructions for directing the image analysis processor 144 to present the medical images according to a pre-defined arrangement of image views. The image analysis tools may include tools for performing measurements, creating annotations, providing diagnosis, and the like. The image analysis tools may be operable to create image objects for association with medical images and/or locations within a medical image. For example, the image objects may include the measurement, annotation, diagnosis, or the like. The image objects may further include an ID or label and a particular location in a particular medical image of the measurement, annotation, diagnosis, or the like.

In this way, the image analysis processor 144 may comprise suitable logic, circuitry, interfaces and/or code that may be operable to provide annotations, measurements, diagnosis, and the like to anatomical structures depicted in medical images presented at the display system 150 in response to user directives provided via the user input device 130 and/or control processor 142. The anatomical structures may include, for example, structures of the heart, lungs, fetus, or any suitable internal body structures. For example, with reference to a heart, a user may provide directives via the user input device 130 and/or control processor 142 to the image analysis processor 144 for annotating or labeling a mitral valve, aortic valve, ventricle chambers, atria chambers, septum, papillary muscle, inferior wall, and/or any suitable heart structure. As another example, a user may provide directives via the user input device 130 and/or control processor 142 to image analysis processor 144 for performing heart measurements, such as a left ventricle internal diameter at end systole (LVIDs) measurement, an interventricular septum at end systole (IVSs) measurement, a left ventricle posterior wall at end systole (LVPWs) measurement, or an aortic valve diameter (AV Diam) measurement, among other things. The user may provide directives via the user input device 130 and/or control processor 142 to image analysis processor 144 for associating a diagnosis with a medical image. For example, the user may dictate findings that may be associated with a medical image and/or location within a medical image. The image analysis processor 144 may superimpose image objects comprising the annotations, measurements, diagnosis, and the like provided via the user input device 130 and/or control processor 142 on the medical image presented at the display system 150 or otherwise associate the image objects comprising the annotations, measurements, diagnosis, and the like with the medical image. For example, each of the image objects associated with the medical images may be stored with or in relation to the associated medical image as metadata. In various embodiments, the metadata may include an ID or label and a set of coordinates corresponding with the location of the image object in the medical image. The image objects having the set of coordinates may be stored at archive 120 and/or at any suitable storage medium.

In a representative embodiment, the image objects may be linked to corresponding report objects created in the report template by the reporting processor 146. The creation or modification of an image object by the image analysis processor 144 is provided to control processor 142 and/or reporting processor 146 to create and/or update a corresponding, linked report object by the reporting processor 146. The creation or modification of a report object by the reporting processor 146 is provided to control processor 142 and/or image analysis processor 144 to create and/or update the corresponding, linked image object by the image analysis processor 144.

In various embodiments, one or more of the image analysis tools of the image analysis processor 144 may include automated analysis features and/or tools that automatically analyze medical images to identify, segment, annotate, perform measurements, provide diagnosis, and/or the like to structures depicted in the medical images. The image analysis processor 144 may include, for example, artificial intelligence image analysis algorithms, one or more deep neural networks (e.g., a convolutional neural network) and/or may utilize any suitable form of artificial intelligence image analysis techniques or machine learning processing functionality configured to provide the automated analysis feature(s) and/or tool(s). For example, one or more of the image analysis tools of the image analysis processor 144 may be provided as a deep neural network that may be made up of, for example, an input layer, an output layer, and one or more hidden layers in between the input and output layers. Each of the layers may be made up of a plurality of processing nodes that may be referred to as neurons. For example, one or more of the image analysis tools may include an input layer having a neuron for each pixel or a group of pixels from a medical of an anatomical structure. The output layer may have a neuron corresponding to a plurality of pre-defined anatomical structures. As an example, if performing an ultrasound-based heart examination, the output layer may include neurons for a mitral valve, aortic valve, ventricle chambers, atria chambers, septum, papillary muscle, inferior wall, and/or any suitable heart structure. Other medical imaging procedures may utilize output layers that include neurons for nerves, vessels, bones, organs, tissue, or any suitable structure. Each neuron of each layer may perform a processing function and pass the processed medical image information to one of a plurality of neurons of a downstream layer for further processing. As an example, neurons of a first layer may learn to recognize edges of structure in the medical image data. The neurons of a second layer may learn to recognize shapes based on the detected edges from the first layer. The neurons of a third layer may learn positions of the recognized shapes relative to landmarks in the medical image data. The processing performed by the image analysis processor 144 deep neural network may identify anatomical structures and the location of the structures in the medical images with a high degree of probability.

The one or more image analysis tools of the image analysis processor 144 may comprise suitable logic, circuitry, interfaces and/or code that may be operable to automatically create image objects comprising annotations, measurements, and/or diagnosis corresponding with the anatomical structures depicted in the medical image. For example, the one or more image analysis tools of the image analysis processor 144 may annotate, measure, and/or diagnose the identified and segmented structures identified by the output layer of the deep neural network. As an example, the one or more image analysis tools of the image analysis processor 144 may be utilized to perform measurements of detected anatomical structures. For example, the one or more image analysis tools of the image analysis processor 144 may be configured to perform a heart measurement, such as a left ventricle internal diameter at end systole (LVIDs) measurement, an interventricular septum at end systole (IVSs) measurement, a left ventricle posterior wall at end systole (LVPWs) measurement, or an aortic valve diameter (AV Diam) measurement. As another example, the one or more image analysis tools may be configured to perform cancer screening measurements, such as a tumor maximum diameter or volume, peak or sum information inside a region of interest, and/or any suitable measurement. The annotations, measurements, and/or diagnosis may be provided in an image object overlaid on the medical image and presented at the display system 150 and/or otherwise associated with the medical image. For example, the image objects associated with the medical images may be stored with or in relation to the associated medical image as metadata. In various embodiments, the metadata may include an ID or label and a set of coordinates corresponding with the location of the image object in the medical image. The image objects having the set of coordinates may be stored at archive 120 and/or at any suitable storage medium.

In various embodiments, the image objects automatically created by one or more image analysis tools of the image analysis processor 144 may be provided to the control processor 142 and/or reporting processor 146 for creating a corresponding report object in the report. The image objects may be linked to corresponding report objects created in the report template by the reporting processor 146. The creation or modification of an image object by the image analysis processor 144 is provided to control processor 142 and/or reporting processor 146 to create and/or update a corresponding, linked report object by the reporting processor 146. The creation or modification of a report object by the reporting processor 146 is provided to control processor 142 and/or image analysis processor 144 to create and/or update the corresponding, linked image object by the image analysis processor 144.

The signal processor 140 may include a reporting processor 146 that comprises suitable logic, circuitry, interfaces and/or code that may be operable to create a report corresponding to the medical image analysis performed by the image analysis processor 144. For example, the reporting processor 146 may retrieve a report template in response to an examination selection provided by a user via the user input device 130 and/or control processor 142. The reporting processor 146 may be configured to create and insert report objects into the report template. The report objects may be created in response to image objects created by the image analysis processor 144 and provided via the image analysis processor 144 and/or control processor 142. Additionally and/or alternatively, the reporting processor 146 may create report objects in response to a user analysis input provided via the user input device 130 and/or control processor 142. For example, a user may dictate a diagnosis or finding to the reporting processor 146 via a dictation user input device 130. The reporting processor 146 may insert the dictation into the report template as a report object. The user may optionally provide additional information, such as an ID, label, a reference to a medical image or location within a medical image, and/or any suitable information, as metadata to the report object via the user input device 130 and/or control processor 142. The report object may be provided to the control processor 142 and/or image analysis processor 144 such that the image analysis processor 144 may create a corresponding, linked image object.

The signal processor 140 may include a control processor 142 that comprises suitable logic, circuitry, interfaces and/or code that may be operable to synchronize object creation and modification by the image analysis processor 144 and reporting processor 146 in response to user analysis inputs received via the user input device 130. For example, the control processor 142 may act on both the image analysis processor 144 and reporting processor 146 to define the nature of an object or diagnostic task that an operator intends to perform. As an example, the control processor 142 may be operable to provide the image analysis processor 144 and reporting processor 146 with an examination selection received via the user input device 130. The examination selection may be used by the image analysis processor 144 to select an appropriate viewing context and may be used by the reporting processor 146 to select an appropriate report template. As another example, the control processor 142 may be operable to provide patient information, examination information, medical professional information, hospital information, and the like to the image analysis processor 144 for associating metadata with the selected medical image data and to the reporting processor 146 for automatically populating fields of the reporting template. In various embodiments, the control processor 142 facilitates the linking of image objects created and/or modified by the image analysis processor 144 with report objects created and/or modified by the reporting processor 146. For example, the control processor 142 is configured to instruct both the image analysis processor 144 and reporting processor 146 based on user inputs received via the user input device 130. The control processor 142 may be configured to provide object linking instructions to the image analysis processor 144 and reporting processor 146 in response to image objects created or modified by the image analysis processor 144. The control processor 142 may be configured to provide object linking instructions to the image analysis processor 144 and reporting processor 146 in response to report objects created or modified by the reporting processor 146.

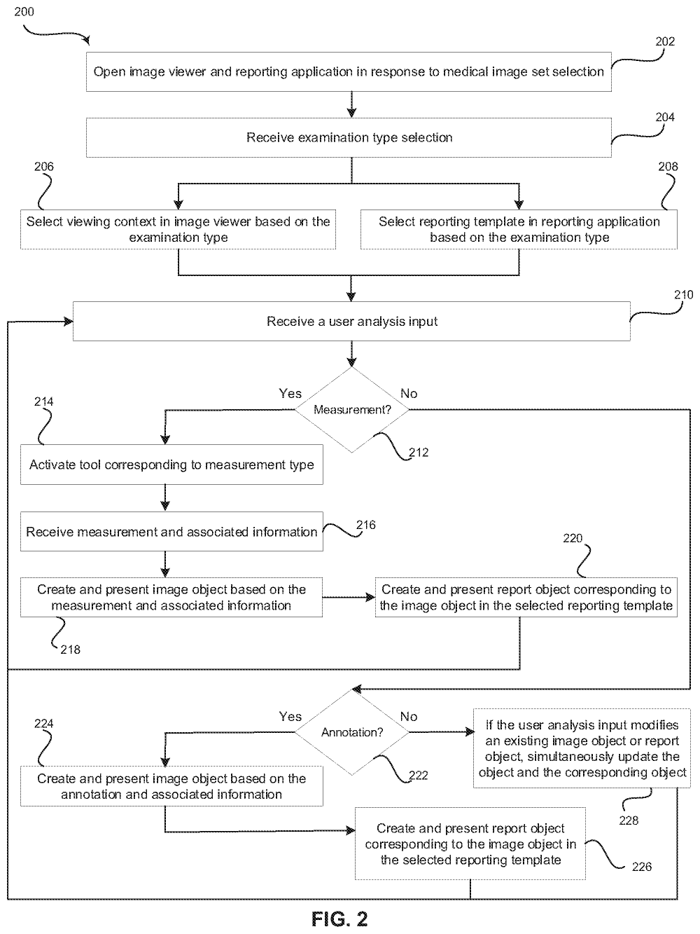

FIG. 2 is a flow chart 200 illustrating exemplary steps 202-228 that may be utilized for synchronizing medical image analysis and reporting, in accordance with various embodiments. Referring to FIG. 2, there is shown a flow chart 200 comprising exemplary steps 202 through 228. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 202, a medical workstation 100 opens an image viewer and reporting application in response to a medical image set selection. For example, a control processor 142 of the signal processor 140 of the medical workstation 100 may receive a user input via the user input device 130 selecting a medical image set for providing analysis and creating a report. The control processor 142 and/or an image analysis processor 144 of the signal processor 140 may retrieve the medical image data set from archive 120 or any suitable data storage medium for presentation at the display system 150 via an image viewer executed by the image analysis processor 144. The control processor 142 may interact with the reporting processor 146 to open the reporting application. The control processor 142 may provide the image analysis processor 144 and/or reporting processor 146 with patient information, medical examination information, medical personnel information, hospital information, and the like for association with the medical image data (e.g., metadata) and the report.

At step 204, the medical workstation 100 may receive an examination type selection. For example, the control processor 142 may receive a user input via use input device 130 selecting an examination type. Additionally and/or alternatively, the control processor 142 may extract the examination type from metadata associated with the selected medical image data set or from automated image analysis. The selected examination type may be provided by the control processor 142 to the image analysis processor 144 and reporting processor 146.

At step 206, the image analysis processor 144 may select a viewing context based on the examination type. For example, the image analysis processor 144 may receive the selected examination type from the control processor 142 at step 204. The image analysis processor 144 may select the viewing context based on the received selection. The viewing context may include a hanging protocol and image analysis tools. The hanging protocol may define a presentation arrangement of the image views from the medical image data set. The image analysis tools may include annotation tools, measurement tools, diagnosis tools, and/or any suitable tools for analyzing the medical image data. In various embodiment, the image analysis tools may include automated analysis tools, such as artificial intelligence tools operable to automatically analyze the medical image data to label, annotate, provide measurements, detect anatomy or abnormal structures, and/or provide diagnosis to the medical images.

At step 208, the reporting processor 146 may select a reporting template based on the examination type. For example, the reporting processor 146 may receive the selected examination type from the control processor 142 at step 204. The reporting processor 146 may select the reporting template based on the received selection. The reporting template may include pre-defined report sections for insertion of patient information, medical examination information, medical personnel information, hospital information, image analysis information, diagnosis information, findings, and/or any suitable report sections. In certain embodiments, the reporting processor 146 may automatically populate sections of the report with information provided by the control processor 142, such as the patient information, medical examination information, medical personnel information, hospital information, and the like, which may correspond with metadata from the medical image data set selected at step 202.

At step 210, the control processor 142 may receive a user analysis input. For example, the control processor 142 may receive a user analysis input via the user input device 130. As an example, the user analysis input may be a measurement instruction provided by a user, an automated measurement provided by the image analysis processor 144, an annotation instruction provided by a user, an automated annotation provided by the image analysis processor 144, an image object modification provided by a user, report object modification provided by a user, and/or any suitable user analysis input.

At step 212, the control processor 142 may determine whether the user analysis input received at step 210 is a measurement. The process 200 may proceed to step 214 if the control processor 142 determines that the user analysis input is a measurement. The process 200 may proceed to step 222 if the control processor 142 determines that the user analysis input is not a measurement.

At step 214, the image analysis processor 144 may activate the tool corresponding to the measurement type. For example, in a lung cancer screening medical examination type, a nodule sizing tool may be activated to measure a size of a nodule depicted in a medical image of the lungs. Other measurement tools may be activated to measure, for example, a tumor maximum diameter or volume, peak or sum information inside a region of interest, and/or any suitable measurement. As another example, in a heart examination, the image analysis processor 144 may activate the tool corresponding to a particular measurement type, such as a left ventricle internal diameter at end systole (LVIDs) measurement, an interventricular septum at end systole (IVSs) measurement, a left ventricle posterior wall at end systole (LVPWs) measurement, or an aortic valve diameter (AV Diam) measurement. The image analysis processor 144 may activate the tool in response to the user analysis input, such as a voice input, a touchscreen selection, or the like. For example, during a lung cancer screening, a user may provide a voice input of “size nodule, left lung, speculated” and the nodule sizing tool may be activated by the image analysis processor 144 in response to the voice input from the user input device 130 via the control processor 142.

At step 216, the image analysis processor 144 may receive a measurement and associated information. For example, the image analysis processor 144 may receive the measurement provided by the tool, such as the nodule sizing tool providing the size of the nodule of the left lung as described above at step 214. The associated information may include the information regarding the medical image from which the measurement was performed (e.g., the medical image of the left lung), a location of the measurement (e.g., a location of the nodule in the left lung medical image), and any other suitable information. In various embodiments, the image analysis processor 144 may prompt the user to provide additional information, such as a label, ID, dictation notes, and/or any suitable information to associate with the measurement. This prompting may be done by instructions on a screen or voice.

At step 218, the image analysis processor 144 may create and present an image object based on the measurement and associated information. For example, the image analysis processor 144 may create an image object providing the label or ID, the associated medical image, a location within the associated medical image, the measurement value, dictation notes, and/or any associated information. The image object may be superimposed at the location on the associated medical image and/or otherwise associated with the medical image and/or annotation location. The medical image having the image object may be presented at the display system 150 of the medical workstation 100.

At step 220, the reporting processor 146 may create and present a report object corresponding to the image object in the selected reporting template. For example, the control processor 142 may provide the image object and/or information from the image object to the reporting processor 146. The reporting processor 146 may create the report object based at least in part on the information from the image object. The report object and image object may be linked and/or otherwise associated. The report object may be inserted into the report template and presented with the report at the display system 150 of the medical workstation 100. In various embodiments, the report template may be presented by the reporting processor 146 simultaneously with the image object in the image viewer at a same or different display of the display system 150. Additionally and/or alternatively, the image viewer and report template may be separately displayed, for example, based on a user instruction to switch between image viewer and report applications. The process may return to step 210 and continue until no further user analysis inputs are received.

At step 222, the control processor 142 may determine whether the user analysis input received at step 210 is an annotation. The process 200 may proceed to step 224 if the control processor 142 determines that the user analysis input is an annotation. The process 200 may proceed to step 228 if the control processor 142 determines that the user analysis input is not an annotation.

At step 224, the image analysis processor 144 may create and present an image object based on an annotation and associated information. For example, the image analysis processor 144 may create an image object providing a label or ID, an associated medical image, a location within the associated medical image, an annotation, and/or any associated information in response to a user analysis input providing an annotation via an annotation tool of the image analysis processor 144. The annotation may be provided, for example, via a dictation device, a keyboard, mousing device, touchscreen display, and/or any suitable user input device 130. The image object may be superimposed at the location on the associated medical image and/or otherwise associated with the medical image and/or annotation location. The medical image having the image object may be presented at the display system 150 of the medical workstation 100.

At step 226, the reporting processor 146 may create and present a report object corresponding to the image object in the selected reporting template. For example, the control processor 142 may provide the image object and/or information from the image object to the reporting processor 146. The reporting processor 146 may create the report object based at least in part on the information from the image object. The report object and image object may be linked and/or otherwise associated. The report object may be inserted into the report template and presented with the report at the display system 150 of the medical workstation 100. The process may return to step 210 and continue until no further user analysis inputs are received.

At step 228, if the user analysis input modifies an existing image object or report object, the modified object and its corresponding linked object may be simultaneously updated. For example, if an annotation or measurement in a report object provided in the report template is modified by a user analysis input via the reporting processor 146, the control processor 142 instructs the image analysis processor 144 to update the corresponding image object in the medical image data set. As another example, if a measurement or annotation in the medical image data set is modified by a user analysis input via the image analysis processor 144, the control processor 142 instructs the reporting processor 146 to update the corresponding report object in the reporting template. Accordingly, a user may modify measurement or annotations in either the image viewer or report and the changes may be simultaneously provided in both the medical image data set and the report. The process may return to step 210 and continue until no further user analysis inputs are received.

Aspects of the present disclosure provide methods 200 and systems 100 for synchronizing medical image analysis and reporting. In accordance with various embodiments, the method 200 may comprise receiving 204, by at least one processor 140, 142, 144, 146 of a medical workstation 100, an examination type selection. The method 200 may comprise selecting 206, by the at least one processor 140, 142, 144, a viewing context from a plurality of viewing contexts applied by an image viewer to a plurality of medical images based on the examination type selection. The method 200 may comprise receiving 210, by the at least one processor 140, 142, 144, 146, a user analysis input with reference to one of the plurality of medical images presented via the image viewer at a display system 150 according to the viewing context. The method 200 may comprise generating 218, 224, by the at least one processor 140, 142, 144, an image object comprising the user analysis input. The image object may be presented via the image viewer at the display system 150 in the one of the plurality of medical images. The method 200 may comprise generating 220, 226, by the at least one processor 140, 142, 146, a report object corresponding to the image object. The report object may comprise the user analysis input. The report object may be inserted in a reporting template.

In a representative embodiment, one or both of the examination type selection and the user analysis input may be a voice input. In an exemplary embodiment, the viewing context may comprise a hanging protocol and image analysis tools. In certain embodiments, the user analysis input may be one or both of a measurement instruction or an annotation provided via at least one of the image analysis tools. In a representative embodiment, the method 200 may comprise selecting 208, by the at least one processor 140, 142, 146, a reporting template from a plurality of reporting templates based on the examination type selection. In various embodiments, the method 200 may comprise modifying 228 the image object in response to an additional user analysis input and automatically updating 228 the report object corresponding to the image object in response to the modifying the image object. In a representative embodiment, the method 200 may comprise modifying 228 the report object in response to an additional user analysis input and automatically updating 228 the image object corresponding to the report object in response to the modifying the report object. In an exemplary embodiment, the image object and the report object may comprise a plurality of an identification label, an identification of the one of the plurality of medical images, a location within the one of the plurality of medical images, and a dictation comment. In certain embodiments, the method 200 may comprise presenting 220, 226, at the display system 150, the reporting template comprising the report object.

Various embodiments provide a system 100 for synchronizing medical image analysis and reporting. The system 100 may comprise a display system 150 and at least one processor 140, 142, 144, 146. The display system 150 may be operable to present a plurality of medical images. The at least one processor 140, 142, 144, 146 may be operable to receive an examination type selection. The at least one processor 140, 142, 144 may be operable to select a viewing context from a plurality of viewing contexts applied by an image viewer to the plurality of medical images based on the examination type selection. The at least one processor 140, 142, 144, 146 may be operable to receive a user analysis input with reference to one of the plurality of medical images presented via the image viewer at the display system 150 according to the viewing context. The at least one processor 140, 142, 144 may be operable to generate an image object comprising the user analysis input. The image object may be presented via the image viewer at the display system 150 in the one of the plurality of medical images. The at least one processor 140, 142, 146 may be operable to generate a report object corresponding to the image object. The report object may comprise the user analysis input. The report object may be inserted in a reporting template.

In an exemplary embodiment, one or both of the examination type selection and the user analysis input may be a voice input. In certain embodiments, the viewing context may comprise a hanging protocol and image analysis tools. The user analysis input may be one or both of a measurement instruction or an annotation provided via at least one of the image analysis tools. In various embodiments, the at least one processor 140, 142, 144, 146 may be operable to modify the image object in response to an additional user analysis input and automatically update the report object corresponding to the image object in response to the modifying the image object. In a representative embodiment, the at least one processor 140, 142, 144, 146 may be operable to modify the report object in response to an additional user analysis input and automatically update the image object corresponding to the report object in response to the modifying the report object. In an exemplary embodiment, the image object and the report object may comprise a plurality of an identification label, an identification of the one of the plurality of medical images, a location within the one of the plurality of medical images, and a dictation comment. In various embodiments, the at least one processor 140, 142, 146 may be operable to select a reporting template from a plurality of reporting templates based on the examination type selection. The at least one processor 140, 142, 146 may be operable to present the reporting template comprising the report object at the display system 150.

Certain embodiments provide a non-transitory computer readable medium having stored thereon, a computer program having at least one code section. The at least one code section is executable by a machine for causing the machine to perform steps 200. The steps 200 may comprise selecting 206 a viewing context from a plurality of viewing contexts applied by an image viewer to a plurality of medical images based on an examination type selection. The steps 200 may comprise receiving 210 a user analysis input with reference to one of the plurality of medical images presented via the image viewer at a display system 150 according to the viewing context. The steps 200 may comprise generating 218, 224 an image object comprising the user analysis input. The image object may be presented via the image viewer at the display system 150 in the one of the plurality of medical images. The steps 200 may comprise generating 220, 226 a report object corresponding to the image object. The report object may comprise the user analysis input. The report object may be inserted in the reporting template.

In various embodiments, the steps 200 may comprise selecting 208 a reporting template from a plurality of reporting templates based on the examination type selection. In certain embodiments, the viewing context may comprise a hanging protocol and image analysis tools. The user analysis input may be one or both of a measurement instruction or an annotation provided via at least one of the image analysis tools. In a representative embodiment, the steps 200 may comprise modifying 228 one of the image object or the report object in response to an additional user analysis input. The steps 200 may comprise automatically updating 228 an other of the report object corresponding to the image object or the image object corresponding to the report object in response to the modifying the one of the image object or the report object. In an exemplary embodiment, the user analysis input may be a voice input. The image object and the report object may comprise a plurality of an identification label, an identification of the one of the plurality of medical images, a location within the one of the plurality of medical images, and a dictation comment.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for synchronizing medical image analysis and reporting.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A medical diagnosis system, comprising:
a display system;
an archive comprising at least one image object;
a user input dictation device with voice recognition capable of receiving and interpreting a user directive;
at least one processor in communication with the display system, user input dictation device, and archive, operable to:
receive an examination type selection via a user directive input from the user input dictation device;
select a report template from a plurality of report templates based on the examination type selection;
link the selected report template with at least one image object;
receive a dictated diagnosis related to the at least one image object via a user directive input from the user input dictation device;
insert the dictated diagnosis into the report object linked with the image object; and
present the report template including the dictated diagnosis, and the linked image object on the display system.

2. The medical diagnosis system of claim 1, wherein the processor is further operable to:
display image analysis tools on the display screen; and
receive a measurement instruction or an annotation provided by the image analysis tools via a user directive input from user input dictation device.

3. The medical diagnosis system of claim 2, wherein the processor is further operable to:
voice prompt a user to provide additional information associated with the measurement or annotation.

4. The medical diagnosis system of claim 1, wherein the processor is further operable to:
superimpose annotations, measurements, or diagnosis information on the image object presented on the display system.

5. The medical diagnosis system of claim 1, wherein:
the archive comprises a Picture Archiving and Communication System (PACS).

6. The medical diagnosis system of claim 1, wherein:
the archive stores medical images, medical image measurements, medical image annotations, report templates, and reports.

7. The medical diagnosis system of claim 1, wherein the processor is further operable to:
modify the image object in response to an additional user directive input from user input dictation device, and
automatically update the report object corresponding to the image object in response to the modifying the image object.

8. The medical diagnosis system of claim 1, wherein the processor is further operable to:
determine if a user directive input from user input dictation device is a measurement related to an image, and if it is a measurement the processor is operable to:
activate a tool corresponding to the measurement type;
receive voice input measurement;
create and present an image object based on the measurement; and
create and present a report object corresponding to the image object in a selected reporting template.

9. The medical diagnosis system of claim 1, wherein the processor is further operable to:
determine if a user directive input from user input dictation device is an annotation related to an image, and if it is a annotation the processor is operable to:
create and present an image object based on the annotation; and
create and present a report object corresponding to the image object in a selected reporting template.

10. A medical diagnosis method performed in a computer system with a display, an archive comprising at least one image object; and a user input dictation device with voice recognition capable of receiving and interpreting a user directive; said method comprising the steps of:
receiving an examination type selection via a user directive input from the user input dictation device;
selecting a report template from a plurality of report templates based on the examination type selection;
linking the selected report template with at least one image object;
receiving a dictated diagnosis related to the at least one image object via a user directive input from the user input dictation device;
inserting the dictated diagnosis into the report object linked with the image object; and
presenting the report template including the dictated diagnosis, and the linked image object on the display system.

11. The medical diagnosis method of claim 10, further comprising the steps of:
displaying image analysis tools on the display screen; and
receiving a measurement instruction or an annotation provided by the image analysis tools via a user directive input from user input dictation device.

12. The medical diagnosis method of claim 11, further comprising the step of:
voice prompting a user to provide additional information associated with the measurement or annotation.

13. The medical diagnosis method of claim 10, further comprising the step of:
superimposing annotations, measurements, or diagnosis information on the image object presented on the display system.

14. The medical diagnosis method of claim 10, wherein:
the archive comprises a Picture Archiving and Communication System (PACS).

15. The medical diagnosis method of claim 10, wherein:
the archive stores medical images, medical image measurements, medical image annotations, report templates, and reports.

16. The medical diagnosis method of claim 10, further comprising the steps of:
modifying the image object in response to an additional user directive input from user input dictation device, and
automatically updating the report object corresponding to the image object in response to the modifying the image object.

17. The medical diagnosis method of claim 10, further comprising the steps of:
determining if a user directive input from user input dictation device is a measurement related to an image, and if it is a measurement the method performs the steps of
activating a tool corresponding to the measurement type;
receiving voice input measurement;
creating and presenting an image object based on the measurement; and
creating and presenting a report object corresponding to the image object in a selected reporting template.

18. The medical diagnosis method of claim 10, further comprising the steps of:
determining if a user directive input from user input dictation device is an annotation related to an image, and if it is a annotation the method performs the steps of
creating and presenting an image object based on the annotation; and
creating and presenting a report object corresponding to the image object in a selected reporting template.
